SEQUENCE LISTING (4 sequences)

SEQ ID NO: 1 - DNA, 1311 bp, Artificial
  Hybrid alpha-amylase BD5088
atggccaagtactccgagctggaaaagggcggggtcataatgcaggcgttctactgggacgtgccttcaggaggaatatggtgggacacaatacggcagaagataccggagtggtacgatgccggaatctccgcaatatggattcccccggcgagcaagggcatgggcggcgcctattcgatgggctacgacccctacgacttctttgacctcggtgagtacgaccagaagggaacggtagagacgcgctttggctccaagcaggagctcgtgaacatgataaacaccgcccacgcctatggcatgaaggtaatagccgatatagtcatcaaccaccgcgccggcggtgacctggagtggaaccccttcgtgaacgactatacctggaccgacttctcaaaggtcgcgtcgggtaaatacacggccaactacctcgacttccacccgaacgagctccatgcgggcgattccggaacatttggaggctatcccgacatatgccacgacaagagctgggaccagtactggctctgggccagccaggagagctacgcggcatatctcaggagcatcggcatcgatgcctggcgcttcgactacgtcaagggctatgctccctgggtcgtcaaggactggctgaactggtggggaggctgggcggttggagagtactgggacaccaacgtcgacgctgttctcaactgggcatactcgagcggtgccaaggtctttgacttcgccctctactacaagatggatgaggcctttgacaacaaaaacattccagcgctcgtctctgcccttcagaacggccagactgttgtctcccgcgacccgttcaaggccgtaacctttgtagcaaaccacgacaccgatataatctggaacaagtatccagcctacgcgttcatcctcacctacgagggccagccgacaatattctaccgcgactacgaggagtggctcaacaaggataagctcaagaacctcatctggatacatgagaacctcgccggaggaagcaccgacatagtctactacgataacgatgaactcatcttcgtcaggaacggctacggggacaagccggggcttataacctacatcaacctaggctcgagcaaggccggaaggtgggtttatgtgccgaagttcgcgggcgcgtgcatccacgagtatactggtaacctcggaggctgggtagacaagtacgtctactcaagcggctgggtctatctcgaagctccagcttacgaccctgccaacgggcagtatggctactccgtgtggagctactgcggggtgggctga

SEQ ID NO: 2 - protein, 436 aa, Artificial
  Synthetic Construct
MAKYSELEKGGVIMQAFYWDVPSGGIWWDTIRQKIPEWYDAGISAIWIPPASKGMGGAYSMGYDPYDFFDLGEYDQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASQESYAAYLRSIGIDAWRFDYVKGYAPWVVKDWLNWWGGWAVGEYWDTNVDAVLNWAYSSGAKVFDFALYYKMDEAFDNKNIPALVSALQNGQTVVSRDPFKAVTFVANHDTDIIWNKYPAYAFILTYEGQPTIFYRDYEEWLNKDKLKNLIWIHENLAGGSTDIVYYDNDELIFVRNGYGDKPGLITYINLGSSKAGRWVYVPKFAGACIHEYTGNLGGWVDKYVYSSGWVYLEAPAYDPANGQYGYSVWSYCGVG

SEQ ID NO: 3 - DNA, 1740 bp, Trametes cingulata
  misc_feature (1)..(1740): cDNA
atgcgtttcacgctcctcacctccctcctgggcctcgccctcggcgcgttcgcgcagtcgagtgcggccgacgcgtacgtcgcgtccgaatcgcccatcgccaaggcgggtgtgctcgccaacatcgggcccagcggctccaagtccaacggagcaaaggcaagtgacaccccggcatcgntgattgcaagtccgagcacatccaacccgaactacctgtacacatggacgcgcgactcgtccctcgtgttcaaggcgctcatcgaccagttcaccactggcgaagatacctcgctccgaactctgattgacgagttcacctcggcggaggccatactccagcaggtgccgaacccgagcgggacagtcagcactggaggcctcggcgagcccaagttcaacatcgacgagaccgcgttcacggatgcctggggtcgtcctcagcgcgatggtcccgctctccgggcgactgccatcatcacctacgccaactggctcctcgacaacaagaacacgacctacgtgaccaacactctctggcctatcatcaagctcgacctcgactacgtcgccagcaactggaaccagtccacgtttgatctctgggaggagattaactcctcgtcgttcttcactaccgccgtccagcaccgtgctctgcgcgagggcgcgactttcgctaatcgcatcggacaaacctcggtggtcagcgggtacaccacccaagcaaacaaccttctctgcttcctgcaggcatcgtactggaaccccaccggcggctatatcaccgcaaacacgggcggcggccgctctggcaaggacgcgaacaccgttctcacgtcgatccacaccttcgacccggccgctggatgcgacgctgttacgttccagccgtgctcggacaaggcgctgtcgaacttgaaggtgtacgtcgatgcgttccgctcgatctactccatcaacagcgggatcgcctcgaatgcggccgttgctaccggccgctaccccgaggacagctacatgggcggaaacccatggtacctcaccacctccgccgtcgctgagcagctctacgatgcgctcattgtgtggaacaagcttggcgccctgaacgtcacgagcacctccctccccttcttccagcagttctcgtcaggcgtcaccgtcggcacctatgcctcatcctcgtccaccttcaagacgctcacttccgccatcaagaccttcgccgacggcttcctcgcggtcaacgccaagtacacgccctcgaacggcggccttgctgaacagtacagccggagcaacggctcgcccgtcagcgctgtggacctgacgtggagctatgctgctgccctcacgtcgtttgctgcgcgctcaggcaagacgtatgcgagctggggcgcggcgggtttgactgtcccgacgacttgctcggggagtggcggtgctgggactgtggccgtcaccttcaacgtgcaggcgaccaccgtgttcggcgagaacatttacatcacaggctcggtccccgctctccagaactggtcgcccgacaacgcgctcatcctctcagcggccaactaccccacttggagcatcaccgtgaacctgccggcgagcacgacgatcgagtacaagtacattcgcaagttcaacggcgcggtcacctgggagtccgacccgaacaactcgatcacgacgcccgcgagcggcacgttcacccagaacgacacctggcggtag

SEQ ID NO: 4 - protein, 579 aa, Trametes cingulata
  misc_feature (43)..(43): The ′Xaa′ at location 43 stands for Met, Val, or Leu.
MRFTLLTSLLGLALGAFAQSSAADAYVASESPIAKAGVLANIGPSGSKSNGAKASDTPASXIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQASYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

(19) United States
(12) Patent Application Publication
(10) Pub. No.: US 2009/0142818 A1
(43) Pub. Date: Jun. 4, 2009

(54) Process of producing a fermentation product

(75) Inventors: Henrik Bisgard-Frantzen, Copenhagen (DK); Kevin S. Wenger, Hanover, NH (US); Michael Trent Elder, Wake Forest, NC (US); Randy Deinhammer, Wake Forest, NC (US); Joyce Aldridge Craig, Pittsboro, NC (US)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/300,140

(22) PCT Filed: May 11, 2007
(86) PCT No.: PCT/US07/68733

§ 371 Date: Nov. 24, 2008

Related U.S. Application Data
(60) Provisional application No. 60/800,220, filed on May 12, 2006.

(51) Int. Cl. C12P 7/06

(52) U.S. Cl. 435/161; 435/41

(57) ABSTRACT

The present invention relates to a process of producing a fermentation product, especially ethanol, from starch-containing material using an alpha-amylase and a carbohydrate-source generating enzyme. The invention also relates to a composition comprising an alpha-amylase and a carbohydrate-source generating enzyme as well as the use such compositions for producing fermentation products.

### CROSS-REFERENCE TO A SEQUENCE LISTING

This application contains a Sequence Listing in computer readable form. The computer readable form is incorporated herein by reference.

### BACKGROUND OF THE INVENTION

1. Field of the Invention

The present invention relates to processes of using an alpha-amylase and a carbohydrate-source generating enzyme for producing a fermentation product, such as ethanol. The invention also relates to a composition comprising combination of alpha-amylase and carbohydrate-source generating enzyme, and the use thereof.

2. Description of the Related Art

A vast number of commercial products, including fermentation products such as alcohols (e.g., ethanol methanol, and butanol) are produced from starch-containing material. Enzymatic processes on gelatinized or un-gelatinized starch-containing material are used widely in industry. Alpha-amylase is used, in conventional liquefaction processes, for thinning the aqueous slurry of gelatinized starch-containing material. Alpha-amylase converts long starch polymers into shorter chains and less viscous dextrins. A carbohydrate-source generating enzyme, such as especially glucoamylase, is then used to convert dextrins into low molecular sugars, e.g., DP₁₋₃, that can be metabolized by a fermenting organism, such as yeast, into the desired fermentation product.

Richardson et al. (The Journal of Biological Chemistry, Vol. 277, No. 29, pp. 267501-26507 (2002)) discloses a chimeric alpha-amylase for starch liquefaction.

WO 2002/38787 discloses a process of producing ethanol including secondary liquefaction step carried out in the presence of a thermostable acid alpha-amylase.

Despite the vast number of processes used and suggested in the art of fermentation product production there is still a need for further improvements.

### SUMMARY OF THE INVENTION

The present invention provides processes for producing fermentation products from gelatinized or un-gelatinized (i.e., uncooked) starch-containing material.

In the first aspect the invention relates to a process for producing a fermentation product from starch-containing material comprising the steps of:
(a) liquefying starch-containing material with an alpha-amylase;
(b) saccharifying the liquefied material using a carbohydrate-source generating enzyme;
(c) fermenting using a fermenting organism.
wherein the alpha-amylase used in liquefaction step (a) is selected from the group consisting of:
   (v) the alpha-amylase shown in SEQ ID NO: 2, or
      i) an allelic variant thereof having alpha-amylase activity; or
      ii) a fragment thereof having alpha-amylase activity:
   (x) an alpha-amylase having an amino acid sequence which has at least 60% identity with amino acids 1 to 435 of SEQ ID NO: 2;
      (y) an alpha-amylase which is encoded by a nucleotide sequence (i) which hybridizes under at least low stringency conditions with nucleotides 4 to 1308of SEQ ID NO: 1, or (ii) a complementary strand of (i);
      (z) a variant comprising a conservative substitution, deletion, and/or insertion of one or more amino acids in positions 1 to 435 of SEQ ID NO: 2.

In the second aspect the invention relates to processes for producing fermentation products from starch-containing material comprising:
(a) saccharifying starch-containing material with an alpha-amylase at a temperature below the initial gelatinization temperature of said starch-containing material,
(b) fermenting using a fermenting organism,
wherein the alpha-amylase used in saccharification step (a) or simultaneous saccharification and fermentation in combined steps (a) and (b) is selected from the group consisting of:
   (v) the alpha-amylase shown in SEQ ID NO: 2, or
      i) an allelic variant thereof having alpha-amylase activity; or
      ii) a fragment thereof having alpha-amylase activity;
   (x) an alpha-amylase having an amino acid sequence which has at least 60% identity with amino acids 1 to 435 of SEQ ID NO: 2;
   (y) an alpha-amylase which is encoded by a nucleotide sequence (i) which hybridizes under at least low stringency conditions with nucleotides 4 to 1308of SEQ ID NO: 1, or (ii) a complementary strand of (i); or
   (z) a variant comprising a conservative substitution, deletion, and/or insertion of one or more amino acids in positions 1 to 435 of SEQ ID NO: 2.

The invention also relates to compositions comprising a carbohydrate-source generating enzyme and an alpha-amylase as described in the “Alpha-Amylase”-section below.

Finally the invention relates to the use of a composition of the invention in processes of the invention.

### DEFINITIONS

Alpha-Amylase activity: The term alpha-amylase (Alpha-1,4-glucan 4 glucanohydrolases, EC 3.2.1.1) is defined as an enzyme which catalyzes hydrolysis of starch and other linear and branched 1,4 glucosidic oligo- and polysaccharides. For purposes of the present invention, alpha-amylase activity is determined according to the procedure described in the ‘Materials & Methods’-section below.

Glucoamylase activity: The term glucoamylase (1,4-alpha-D-glucan glucohydrolase, EC 3.2.1.3) is defined as an enzyme, which catalyzes the release of D-glucose from the non-reducing ends of starch or related oligo- and polysaccharide molecules. For purposes of the present invention, glucoamylase activity is determined according to the procedure described in the ‘Materials & Methods’-section below.

Identity: The related ness between two amino acid sequences or between two nucleotide sequences is described by the parameter “identity”.

For purposes of the present invention, the degree of identity between two amino acid sequences is determined by the Clustal method (Higgins, 1989, CABIOS 5:151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=1, gap penalty=3, windows=5, and diagonals=5.

For purposes of the present invention, the degree of identity between two nucleotide sequences is determined by the Wilbur-Lipman method (Wilbur and Lipman. 1983, Proceedings of the National Academy of Science USA 80:726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

Subsequence: The term “subsequence” is defined herein as a nucleotide sequence having one or more nucleotides deleted from the 5′ and/or 3′ end of SEQ ID NO: 1, or homologous sequences thereof, wherein the subsequence encodes an alpha-amylase.

Fragment: The term “fragment” is defined herein as a polypeptide having one or more amino acids deleted from the amino and/or carboxyl terminus of SEQ ID NO: 2, or homologous sequences thereof, wherein the fragment has glucoamylase activity.

Allelic variant: The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Artificial variant: When used herein, the term “artificial variant” means a polypeptide having alpha-amylase activity produced by an organism expressing a modified nucleotide sequence of SEQ ID NO: 1. The modified nucleotide sequence is obtained through human intervention by modification of the nucleotide sequence disclosed in SEQ ID NO: 1.

### DETAILED DESCRIPTION OF THE INVENTION

### Production of Fermentation Products

Processes for Producing Fermentation Products from Gelatinized Starch-Containing Material

In this aspect the present invention relates to a process for producing a fermentation product, especially ethanol, from starch-containing material, which process includes a liquefaction step and sequentially or simultaneously performed saccharification and fermentation steps.

According to the present invention fermentation products, such as ethanol, may advantageously be produced using an alpha-amylase referred to below in combination with a carbohydrate-source generating enzyme. Due to the thermostability of the alpha-amylase the enzyme action time during liquefaction would be prolonged at a pH around 5.6 and below. Further, when combining said alpha-amylase with a carbohydrate-source generating enzyme, especially glucoamylase, a more robust production process is obtained.

Therefore, in the first aspect the invention relates to a process for producing a fermentation product from starch-containing material comprising the steps of:
(a) liquefying starch-containing material with an alpha-amylase;
(b) saccharifying the liquefied material using a carbohydrate-source generating enzyme;
(c) fermenting using a fermenting organism.
wherein the alpha-amylase used in liquefaction step (a) is selected from the group consisting of:
   (v) the alpha-amylase shown in SEQ SD NO: 2, or
      i) an allelic variant thereof having alpha-amylase activity, or
      ii) a fragment thereof having alpha-amylase activity,
   (x) an alpha-amylase having an amino acid sequence which has at least 60% identity with amino acids 1 to 435 of SEQ ID NO: 2;
   (y) an alpha-amylase which is encoded by a nucleotide sequence (i) which hybridizes under at least low stringency conditions with nucleotides 4 to 1308 of SEQ ID NO: 1, or (ii) a complementary strand of (i); or
   (z) a variant comprising a conservative substitution, deletion, and/or insertion of one or more amino acids in positions 1 to 435 of SEQ ID NO: 2.

In a preferred embodiment one or more carbohydrases, especially a second alpha-amylase or a pullulanase, or a combination thereof, may be introduced at step (a). According to the invention a second alpha-amylase may be present during liquefaction in step (a). The second alpha-amylase may be of bacterial or fungal origin, preferably an acid alpha-amylase, especially acid fungal alpha-amylase; of plant origin, such as of corn, wheat or barley origin. Examples of contemplated second alpha-amylases and pullulanases are described below in the section “Additional Enzymes”.

The fermentation product, especially ethanol, may optionally be recovered after fermentation, e.g., by distillation. Suitable starch-containing starting materials are listed in the section “Starch-Containing Materials”-section below. Contemplated enzymes are listed in the “Enzymes”-section below. The liquefaction step is carried out in the presence of an alpha-amylase as defined in above and in the “Alpha-Amylases”-section below. In a preferred embodiment the carbohydrate-source generating enzyme used for saccharification step (b), or combined steps (b) and (c) (i.e., SSF), is a glucoamylase. The fermentation step (c), or combined/simultaneous steps (b) and (c), are preferably carried out in the presence of yeast, preferably a strain of Saccharomyces, such as Saccharomyces cerevisae. Suitable fermenting organisms are listed in the “Fermenting Organisms”-section below. In a preferred embodiment step (b) and (c) are carried out simultaneously (i.e., as SSF). Because of the properties of the alpha-amylase used no calcium ions need to be added during liquefaction.

In a particular embodiment, the process of the invention further comprises, prior to the step (a), the steps of:

1) reducing the particle size of the starch-containing material, preferably by milling;

2) forming a slurry comprising the starch-containing material and water.

The aqueous slurry may contain from 10-55 wt-%, preferably 25-40 wt-%, more preferably 30-35 wt-% starch-containing material. The slurry may be heated to above the gelatinization temperature and alpha-amylase may be added to initiate liquefaction (thinning). The slurry may in one embodiment be jet-cooked to further gelatinize the slurry before being subjected to an alpha-amylase in step (a) of the invention.

More specifically liquefaction may in one embodiment be carried out as a three-step hot slurry process. The slurry is heated to between 60-105° C., preferably 80-95° C., and alpha-amylase may be added to initiate liquefaction (thinning). In one embodiment the slurry is then jet-cooked at a temperature between 95-140° C., preferably 105-125° C., for 1-15 minutes, preferably for 3-10 minute, especially around 5 minutes. The slurry is cooled to 60-105° C. and more alpha-amylase is added to finalize hydrolysis (secondary liquefaction). The liquefaction process may be carried out at a pH from 3-7, in particular at a pH between 4-6, especially at a pH between 4-5. If is to be understood that an alpha-amylase may be added as a single dose, e.g., before jet-cooking.

The saccharification in step (b) may be carried out using conditions well known in the art. For instance, a full saccharification process may last up to from about 24 to about 72 hours. In one embodiment a pre-saccharification of typically 40-90 minutes at a temperature between 30-65° C., typically about 60° C., is carried out, followed by complete saccharification during fermentation in a simultaneous saccharification and fermentation process (i.e., SSF), Saccharification is typically carried out at temperatures from 30-65° C., typically around 60° C., and at a pH between 4 and 5, normally at about pH 4.5.

The most widely used process in fermentation product, especially ethanol, production is a simultaneous saccharification and fermentation (SSF) process, in which there is no holding stage for the saccharification, meaning that the fermenting organism, such as yeast, and enzyme(s) may be added together. SSF may typically be carried out at a temperature between 25° C. and 40° C., such as between 29° C. and 35° C., such as between 30° C. and 34° C., such as around 32° C. According to the invention the temperature may be adjusted up or down during fermentation.

Processes for Producing Fermentation Products from Un-Gelatinized Starch-Containing

In this aspect the invention relates to processes for producing a fermentation product from starch-containing material without cooking (i.e., no gelatinization) of the starch-containing material. According to the invention a desired fermentation product, such as ethanol, may be produced without liquefying the aqueous slurry containing the starch-containing material, in one embodiment a process of the invention includes saccharifying (e.g., milled) starch-containing material, e.g., granular starch, below the initial gelatinization temperature in the presence of an alpha-amylase as defined in the “Alpha-Amylase”-section below; and further a carbohydrate-source generating enzyme, preferably a glucoamylase, disclosed in the “Carbohydrate-Source Generating Enzymes”-section below, to produce sugars that can be fermented and/or converted into a desired fermentation product by a suitable fermenting organism.

Accordingly, in this aspect the invention relates to a process for producing a fermentation product from starch-containing material comprising:
(a) saccharifying starch-containing material with an alpha-amylase at a temperature below the initial gelatinization temperature of said starch-containing material,
(b) fermenting using a fermenting organism,
wherein the alpha-amylase used in saccharification step (a) or simultaneous saccharification and fermentation in combined step (a) and (b) is selected from the group consisting of:
   (v) the alpha-amylase shown in SEQ ID NO: 2, or
      i) an allelic variant thereof having alpha-amylase activity; or
      ii) a fragment thereof having alpha-amylase activity;
   (x) an alpha-amylase having an amino acid sequence which has at least 60% identity with amino acids 1 to 435 of SEQ ID NO: 2;
   (y) an alpha-amylase which is encoded by a nucleotide sequence (i) which hybridizes under at least low stringency conditions with nucleotides 4 to 1308 of SEQ ID NO: 1, or (ii) a complementary strand of (i); or
   (z) a variant comprising a conservative substitution, deletion, and/or insertion of one or more amino acids in positions 1 to 435 of SEQ ID NO: 2.

In one embodiment an acid alpha-amylase, such as an acid fungal alpha-amylase, or a plant alpha-amylase is also added during saccharification or fermentation or simultaneous steps (a) and (b). The additional alpha-amylase may be derived from a bacteria or fungal cell, such as a filamentous fungus. Examples of additional alpha-amylases, preferably acid alpha-amylases, are described in the “Additional Enzymes”-section below.

Steps (a) and (b) of the process of the invention may be carried out sequentially or simultaneously. The fermentation product may be recovered after fermentation.

The term “ . . . below the initial gelatinization temperature . . . ” means the lowest temperature at which gelatinization of the starch in question commences. Starch heated in water begins to gelatinize between about 50° C. and 75° C.: the exact temperature of gelatinization depends on the specific starch and can readily be determined by the skilled artisan. Thus, the initial gelatinization temperature may vary according to the plant species, to the particular variety of the plant species as well as with the growth conditions, in the context of this invention the initial gelatinization temperature of a given starch-containing material can be defined as the temperature at which birefringence is lost in 5% of the starch granules using the method described by Gorinstein. S. and Lii. C, Starch/Stärke, Vol. 44 (12) pp. 461-466 (1992).

Before step (a) a slurry of starch-containing material, such as granular starch, having between 10-55 wt-% dry solids (DS), preferably between 25-40 wt-% dry solids, more preferably 30-35 wt-% dry solids of starch-containing material, may be prepared. The slurry may include water and/or process water, such as thin stillage (backset), scrubber water, evaporator condensate or distillate, side stripper water from distillation, or other fermentation product plant process water. Because the process is carried out below the initial gelatinization temperature and thus no significant viscosity increase takes place, high levels of stillage may be used if desired, in an embodiment the aqueous slurry contains from about 1 to about 70 vol.-% stillage, preferably 15-60 vol.-% stillage, especially from about 30 to 50 vol.-% stillage.

The starch-containing material may be prepared by reducing the particle size, preferably by dry or wet milling, to between 0.05 to 3.0 mm, preferably between 0.1-0.5 mm. After being subjected to a process of the invention at least 60%, at least 70%, at least 80%, at least 90%, at least 90%, at least 91%, at least 92%, at least 93%, at least 94%, at least 95%, at least 96%, at least 97%, at least 98%, or preferably at least 99% of the dry solids of the starch-containing material is converted into a soluble starch hydrolysate.

The process of the invention is conducted at a temperature below the initial gelatinization temperature. Preferably the temperature at which step (a) is carried out sequentially is between 50-75° C., preferably between 45-60° C. In a preferred embodiment step (a) and step (b) are carried out as a simultaneous saccharification and fermentation process (i.e., one-step fermentation), in such preferred embodiment the process may typically be carried out at temperatures between 25° C. and 40° C., such as between 29° C. and 35° C., such as between 30° C. and 34° C., such as around 32° C. According to the invention the temperature may be adjusted up or down during fermentation.

In an embodiment steps (a) and (b) are carried out simultaneously (i.e., one-step fermentation) so that the sugar level, such as glucose level, is kept at a low level such as below 6 wt.-%, preferably below about 3 wt.-%, preferably below about 2 wt.-%, more preferred below about 1 wt-% even more preferred below about 0.5%, or even more preferred 0.25% wt.-%, such as below about 0.1 wt.-%. Such low levels of sugar can be accomplished by simply employing adjusted quantities of enzyme and fermenting organism. A skilled person in the art can easily determine which quantities of enzyme and fermenting organism to use. The employed quantities of enzyme and fermenting organism may also be selected to maintain low concentrations of maltose in the fermentation broth. For instance, the maltose level may be kept below about 0.5 wt.-% or below about 0.2 wt.-%.

The process of the invention may be carried out at a pH in the range between 3-7, preferably from pH 3-6, or more preferably from pH 4-5.

Any suitable starch-containing starting material, including granular starch, may be used according to the present invention. As indicated above the starch-containing material may either be gelatinized or un-gelatinized (i.e., uncooked).

The actual starting material is generally selected based on the desired fermentation product. Examples of starch-containing starting materials, suitable for use in a process of present invention, include tubers, roots, stems, whole grains, corns, cobs, wheat, barley, rye, milo, sago, cassaya, tapioca, sorghum, rice peas, beans, or sweet potatoes, or mixtures thereof, or cereals, sugar-containing raw materials, such as molasses, fruit materials, sugar cane or sugar beet, potatoes, and cellulose-containing materials, such as wood or plant residues, or mixtures thereof. Contemplated are both waxy and non-waxy types of corn and barley.

The term “granular starch” means raw uncooked starch, i.e., starch in its natural form found in cereal, tubers or grains. Starch is formed within plant cells as tiny granules insoluble in water. When put in cold water, the starch granules may absorb a small amount of the liquid and swell. At temperatures up to 50° C. to 75° C. the swelling may be reversible. However, with higher temperatures an irreversible swelling called “gelatinization” begins. Granular starch to be processed may be a highly refined starch quality, preferably at least 90%, at least 95%, at least 97% or at least 99.5% pure or it may be a more crude starch containing material comprising milled whole grain including non-starch fractions such as germ residues and fibers.

### Fractionation of Starch-Containing Material

In an embodiment the starch-containing material is fractionated into one or more components, including fiber, germ, and a mixture of starch and protein (endosperm). Fractionation may according to the invention be done using any suitable technology or apparatus. For instance, Satake Corporation (Japan), has manufactured a system suitable for fractionation of plant material such as corn.

The germ and fiber components may be fractionated from the remaining portion of the endosperm. In an embodiment of the invention the starch-containing material is plant endosperm, preferably corn endosperm. Further, the endosperm may be reduced in particle size and combined with the larger pieces of the fractionated germ and fiber components for fermentation.

Fractionation can be accomplished, e.g., using the apparatus disclosed in US application publication no. 2004/0043117 (hereby incorporated by reference). Suitable methods and apparatus for fractionation include a sieve, sieving and elutriation. Suitable apparatus also include friction mills, such as rice or grain polishing mills (e.g. those manufactured by Satake Corporation (Japan), Kett, or Rapsco, Tex., USA).

### Reducing the Particle Size of Starch-Containing Plant Material

The starch-containing material such as whole grain, used in a process of the invention, may preferably be reduced in particle size in order to open up the structure and expose more surface area. This may be done by milling. Two milling processes are preferred according to the invention: wet and dry milling. In dry milling whole kernels are milled and used. Wet milling gives a good separation of germ and meal (starch granules and protein) and is often applied at locations where the starch hydrolysate is used in production of syrups. Both dry and wet milling is well known in the art of starch processing and is equally contemplated for the process of the invention. Examples of other contemplated technologies for reducing the particle size of the starch-containing plant material include emulsifying technology and rotary pulsation.

The starch-containing material may be reduced in particle size to between 0.05 to 3.0 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen.

### Fermentation Products

The term “fermentation product” means a product produced by a process including a fermentation step using a fermenting organism. Fermentation products contemplated according to the invention include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or Sight beer. Preferred fermentation processes used include alcohol fermentation processes, as are well known in the art. Preferred fermentation processes are anaerobic fermentation processes, as are well known in the art.

### Fermenting Organism

“Fermenting organism” refers to any organism, including bacterial and fungal organisms, suitable for use in a fermentation process and capable of producing desired a fermentation product, Especially suitable fermenting organisms are able to ferment, i.e., convert, sugars, such as glucose or maltose, directly or indirectly into the desired fermentation product. Examples of fermenting organisms include fungal organisms, such as yeast. Preferred yeast includes strains of Saccharomyces spp., in particular, Saccharomyces cerevisiae. Commercially available yeast include, e.g., RED STAR™/Lesaffre. ETHANOL RED™ (available from Red Star/Lesaffre, USA) FALI (available from Fleischmann's Yeast, a division of Burns Philp Food Inc., USA), SUPERSTART (available from Alltech), GERT STRAND (available from Gert Strand AB, Sweden) and FERMIOL™ (available from DSM Specialties).

### Enzymes

### Alpha-Amylase

The alpha-amylase used in a process of the invention may be an alpha-amylase selected from the group consisting of:

(v) the alpha-amylase shown in SEQ ID NO: 2, or
i) an allelic variant thereof having alpha-amylase activity, or
ii) a fragment thereof having alpha-amylase activity,

(x) the alpha-amylase having an amino acid sequence which has at least 60% identity with amino acids 1 to 435 of SEQ ID NO: 2;

(y) the alpha-amylase which is encoded by a nucleotide sequence (i) which hybridizes under at least low stringency conditions with nucleotides 4 to 1308 of SEQ SD NO: 1, or (ii) a complementary strand of (i);

(z) a variant comprising a conservative substitution, deletion, and/or insertion of one or more amino acids in positions 1 to 435 of SEQ ID NO: 2.

In a preferred embodiment the alpha-amylase is the mature part of the alpha-amylase disclosed in Richardson et al, (The Journal of Biological Chemistry, Vol. 277, No 29, pp. 267501-28507 (2002)), referred to as BD5088. This alpha-amylase is the same as the one shown in SEQ ID NO: 2. The mature enzyme sequence starts after the initial “Met” amino acid in position 1.

In a preferred embodiment the alpha-amylase used in a process of the invention is derived from a microorganism, preferably a bacterium, of the order Thermococcales. The alpha-amylase may be a hybrid alpha-amylase such as the BD5088 alpha-amylase made from alpha-amylases from three microorganisms within the order Thermococcales.

The alpha-amylases may according to the process of the invention be added in an amount of 0.1 to 10 AFAU/g DS, preferably 0.10 to 5 AFAU/g DS, especially 0.3 to 2 AFAU/g DS. When measured in KNU units the alpha-amylase activity is preferably present in an amount of 0.0005-5 KNU per g DS, preferably 0.001-1 KNU per g DS, such as around 0.050 KNU per g DS.

In a preferred embodiment the alpha-amylase is the commercially available product sold as ULTRA THIN™ (Valley Research, USA).

### Hybridization

The alpha-amylase used in a process of the invention may in one embodiment be encoded by polynucleotides (i) which hybridizes under at least low stringency conditions, preferably medium stringency conditions, more preferably medium-high stringency conditions, even more preferably high stringency conditions, and most preferably very high stringency conditions with a nucleotide sequence with nucleotides 4 to 1308 of SEQ ID NO: 1, or (ii) a subsequence of (i), or (iii) a complementary strand of (i) or (ii) (J. Sambrook, E. F. Fritsch, and T. Maniatis, 1989, Molecular Cloning, A Laboratory Manual, 2d edition, Cold Spring Harbor, N.Y.). A subsequence of SEQ ID NO: 1 contains at least 100 contiguous nucleotides or preferably at least 200 contiguous nucleotides.

The nucleotide sequence of SEQ ID NO: 1, or a subsequence thereof, as well as the amino acid sequence of SEQ SD NO: 2, or a fragment thereof, may be used to design a nucleic acid probe to identify and clone DNA encoding polypeptides having alpha-amylase activity from strains of different genera or species of especially the order Thermococcales according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes which are at least 600 nucleotides, at least preferably at least 700 nucleotides, more preferably at least 800 nucleotides, or most preferably at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other organisms may, therefore, be screened for DNA which hybridizes with the probes described above and which encodes a polypeptide having alpha-amylase activity. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with SEQ ID NO: 1, or a subsequence thereof, the carrier material is used in a Southern blot.

For long probes of at least 100 nucleotides in length, low to very high stringency conditions are defined as prehybridization and hybridization at 42° C. in 5×SSPE, 0.3% SDS, 200 micro g/ml sheared and denatured salmon sperm DNA, and either 25% formamide for low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC. 0.2% SDS preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6. 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

Under salt-containing hybridization conditions, the effective Tₘ is what controls the degree of identity required between the probe and the filter bound DNA for successful hybridization. The effective Tₘ may be determined using the formula below to determine the degree of identity required for two DNAs to hybridize under various stringency conditions.

Effective Tₘ=81.5+16.6(log M[Na⁺])+0.41(% G+C)−0.72(% formamide)

(See www.ndsu.nodak.edu/instruct/mcclean/plsc731/dna/dna6.htm)

### Variants or Fragments

As mentioned above, the alpha-amylase used in a process of the invention may be a variant of the alpha-amylase shown in SEQ ID NO. 2. A variant may be an allelic or an artificial variant, including a fragment having alpha-amylase activity, in an embodiment of the invention the variant is an artificial variant comprising a conservative substitution, deletion, and/or insertion in positions 1-435 of SEQ ID NO: 2. Preferably, amino acid changes are of a minor nature, that is conservative amino acid substitutions or insertions that do not significantly affect the folding and/or activity of the protein: small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine. 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethyl proline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in the parent polypeptide can be identified according to procedures known in the art, such as site-directed mutagenesis or alanine-scanning mutagenesis (Cunningham and Welts, 1989, Science 244:1081-1085). In the latter technique, single alanine mutations are introduced at every residue in the molecule, and the resultant mutant molecules are tested for biological activity (i.e., glucoamylase activity) to identify amino acid residues that are critical to the activity of the molecule. See also, Hilton et al., 1996, J, Biol. Chem. 271:4699-4708. The active site of the enzymes or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identities of essential amino acids can also be inferred from analysis of identities with polypeptides which are related to a polypeptide according to the invention.

Single or multiple amino acid substitutions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PGR, phage display (e.g., Lowman et al. 1991, Biochem. 30:10832-10837; U.S. Pat. No. 5,223,409; WO 92/06204), and region-directed mutagenesis (Derbyshire et al., 1986, Gene 46:145: Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The total number of amino acid substitutions, deletions and/or insertions of amino acids in position 1 to 558 of SEQ ID NO: 2, is 10, preferably 9, more preferably 8, more preferably 7, more preferably at most 6, more preferably at most 5, more preferably 4, even more preferably 3, most preferably 2, and even most preferably 1.

### Carbohydrate-Source Generating Enzyme

The term “carbohydrate-source generating enzyme” includes glucoamylase (being glucose generators), beta-amylase and maltogenic amylase (being maltose generators). A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol.

According to the invention a combination or mixture of carbohydrate-source generating enzyme and alpha-amylase may be used in a process of the invention. Especially contemplated combinations or mixtures are include one or more glucoamylases as disclosed below in the “Glucoamylases”-section and an alpha-amylase as defined in the Alpha-Amylase”-section below. The ratio between alpha-amylase activity (AFAU) per glucoamylase activity (AGU) (AFAU per AGU) may in an embodiment of the invention be at least 0.1, in particular at least 0.16, such as in the range from 0.12 to 0.50 or more.

### Glucoamylases

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al., 1984, EMBO J. 3(5): 1097-1102), or variants thereof, such as those disclosed in WO 92/00381, WO 00/04136 and WO 01/04273 (from Novozymes, Denmark); the A. awamori glucoamylase disclosed in WO 84/02921, A. oryzae glucoamylase (Agric. Biol, Chem., 1991, 55(4): 941-949), or variants or fragments thereof. Other Aspergillus glucoamylase variants include variants with enhanced thermal stability: G137A and G139A (Chen et al., 1996, Prot. Eng. 9:499-505); D257E and D293E/Q (Chen et al., 1995, Prot. Eng. 8:575-582): N182 (Chen et al. (1994), Biochem. J. 301:275-281); disulphide bonds, A246C (Fierobe et al., 1996, Biochemistry, 35:8698-8704; and introduction of Pro residues in position A435 and S436 (Li et al., 1997, Protein Eng. 10; 1199-1204.

Other glucoamylases include Athelia rolfsii (previously denoted Corticium rolfsii) glucoamylase (see U.S. Pat. No. 4,727,026 and Nagasaka et al., 1998, “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamylolyticum (EP 135,138), and C. thermohydrosulfuricum (WO 86/01831) and Trametes cingutata disclosed in WO 2006/069289, and disclosed in SEQ ID NO: 4 herein (which reference is hereby incorporated by reference).

Also hybrid glucoamylase are contemplated according to the invention. Examples the hybrid glucoamylases disclosed in WO 2005/045018. Specific examples include the hybrid glucoamylase disclosed in Table 1 and 4 of Example 1 (which hybrids are hereby incorporated by reference.).

Preferred glucoamylases are the glucoamylase is selected from the group consisting of glucoamylases derived from the genus Aspergillus, preferably a strain of Aspergillus niger, Aspergillus oryzae, Aspergillus awamori, or the genus Athelia, preferably a strain of Athelia rolfsii, the genus Talaromyces, preferably a strain the Talaromyces emersonii, or the genus Rhizopus, such as a strain of Rhizopus nivius, or of the genus Humicola, preferably a strain of Humicola grisea var. thermoidea, or a strain of the genus Trametes, preferably a strain of Trametes cingulata disclosed in co-pending application PCT/US05/46724 which is hereby incorporated by reference.

Glucoamylases may in an embodiment be added in an amount of 0.001 to 10 AGU/g DS, preferably from 0.01 to 5 AGU/g DS, especially 0.1 to 0.5 AGU/g DS.

### Beta-Amylase

At least according to the invention the a beta-amylase (E.C. 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose re-leased has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.6 to 7. A commercially available beta-amylase from barley is NOVOZYM™ WBA from Novozymes A/S, Denmark and SPEZYME™ BBA 1500 from Genencor Int., USA.

### Maltogenic Amylase

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference.

The maltogenic amylase may in a preferred embodiment be added in an amount of 0.05-5 mg total protein/gram DS or 0.05-5 MANU/g DS.

### Additional Enzymes

As mentioned above, processes of the invention, both non-cook processes (i.e., un-gelatinized starch processes) and gelatinized starch processes (i.e., including a liquefaction step) may in preferred embodiments include introduction of one or more additional carbohydrases, especially alpha-amylases and/or pullulanases.

### Alpha-Amylase

Contemplated additional alpha-amylases may be any alpha-amylase. Preferred are alpha-amylases of fungal or bacterial origin. The alpha-amylase may also be of plant origin, preferably corn, wheat or barley origin.

In a preferred embodiment the additional alpha-amylase is an acid alpha-amylase. The acid alpha-amylase may be of fungal or bacterial origin. The term “acid alpha-amylase” means an alpha-amylase (E.C. 3.2.1.1) which added in an effective amount has activity optimum at a pH in the range of 2 to 7, preferably from 3 to 6, or more preferably from 3.5-5.5.

### Bacterial Alpha-Amylases

A bacterial alpha-amylase may preferably be derived from the genus Bacillus.

In a preferred embodiment the Bacillus alpha-amylase is derived from a strain of B. licheniformis, S. amyloliquefaciens, B. subtilis or S. stearothermophilus, but may also be derived from other Bacillus sp. Specific examples of contemplated alpha-amylases include the Bacillus licheniformis alpha-amylase (BLA) shown in SEQ ID NO: 4 in WO 99/19467, the Bacillus amyloliquefaciens alpha-amylase (BAN) shown in SEQ ID NO: 5 in WO 99/19467, and the Bacillus stearothermophilus alpha-amylase (BSG) shown in SEQ ID NO: 3 in WO 99/19467, in an embodiment of the invention the alpha-amylase is an enzyme having a degree of identity of at least 60%, preferably at least 70%, more preferred at least 80%, even more preferred at least 90%, such as at least 95%, at least 96%, at least 97%, at least 98% or at least 99% identity to any of the sequences shown as SEQ ID NOS: 1, 2, 3, 4, or 5, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038or U.S. Pat. No. 6,187,576 (hereby incorporated by reference) and include Bacillus stearothermophilus alpha-amylase (BSC alpha-amylase) variants having a deletion of one or two amino acid in position 179 to 182, preferably a double deletion disclosed in WO 1996/023873—see e.g., page 20, lines 1-10 (hereby incorporated by reference), preferably corresponding to delta (181-182) compared to the wild-type BSG alpha-amylase amino acid sequence set forth in SEQ ID NO: 3 disclosed in WO 99/19467 or deletion of amino acids 179 and 180 using SEQ ID NO: 3 in WO 99/19467 for numbering (which reference is hereby incorporated by reference). Even more preferred are Bacillus alpha-amylases, especially Bacillus stearothermophilus alpha-amylase, which have a double deletion corresponding to delta (181-182) and further comprise a N193F substitution (also denoted 1181*+G182*+N193F) compared to the wild-type BSG alpha-amylase amino acid sequence set forth in SEQ ID NO: 3 disclosed in WO 99/19467.

The alpha-amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic alpha-amylase from Bacillus stearothermophilus strain NCIB 11837 is commercially available from Novozymes A/S, Denmark. The maltogenic alpha-amylase is described in U.S. Pat. Nos. 4,598,048, 4,604,355 and 6,162,628, which are hereby incorporated by reference.

Other bacterial alpha-amylases contemplated may be derived from Pyrococcus sp., such as Pyrococcus furiosus, such as the ones disclosed in WO 94/19454 which is hereby incorporated by reference.

### Bacterial Hybrid Alpha-Amylases

A hybrid alpha-amylase specifically contemplated comprises 445 C-terminal amino acid residues of the Bacillus licheniformis alpha-amylase (shown as SEQ ID NO: 4 in WO 99/19467) and the 37 N-terminal amino acid residues of the alpha-amylase derived from Bacillus amyloliquefaciens (shown as SEQ ID NO: 3 in WO 99/194676), with one or more, especially all, of the following substitution:

G48A+T49I+G107A+H156Y+A181T+N190F+I201F+A209V+Q264S (using the Bacillus licheniformis numbering). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 99/19467).

The bacterial alpha-amylase may be added in amounts well-known in the art.

### Fungal Alpha-Amylases

Acid fungal alpha-amylases include acid alpha-amylases derived from a strain of the genus Aspergillus, such as Aspergillus oryzae, Aspergillus niger and Aspergillus kawachii.

A preferred acid fungal alpha-amylase is a Fungamyl-like alpha-amylase which is preferably derived from a strain of Aspergillus oryzae, in the present disclosure, the term “Fungamyl-like alpha-amylase” indicates an alpha-amylase which exhibits a high identity, i.e. more than 70%, more than 75%, more than 80%, more than 85% more than 90%, more than 95%, more than 96%, more than 97%, more than 98%, more than 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from A. niger disclosed as “AMYA_ASPNG” in the Swiss-prot/TeEMBL database under the primary accession no. P56271 and described in more detail in WO 89/01969 (Example 3). The acid Aspergillus niger acid alpha-amylase is also shown as SEQ ID NO: 1 in WO 2004/080923 (Novozymes) which is hereby incorporated by reference. Also variants of said acid fungal amylase having at least 70% identity, such as at least 80% or even at least 90% identify, such as at least 95%, at least 96%, at least 97%, at least 98%, or at least 99% identity to SEQ ID NO: 1 in WO 2004/080923 are contemplated. A suitable commercially available acid fungal alpha-amylase derived from Aspergillus niger is SP288 (available from Novozymes A/S, Denmark).

In a preferred embodiment the alpha-amylase is derived from Aspergillus kawachii and disclosed by Kaneko et al. (J. Ferment. Bioeng. 81:292-298 (1996) “Molecular-cloning and determination of the nucleotide-sequence of a gene encoding an acid-stable alpha-amylase from Aspergillus kawachii”) and further as EMBL:#AB008370.

The fungal acid alpha-amylase may also be a wild-type enzyme comprising a carbohydrate-binding module (CBM) and an alpha-amylase catalytic domain (i.e. a none-hybrid), or a variant thereof. In an embodiment the wild-type acid alpha-amylase is derived from a strain of Aspergillus kawachii.

### Fungal Hybrid Alpha-Amylases

In a preferred embodiment the fungal acid alpha-amylase is a hybrid alpha-amylase. Preferred examples of fungal hybrid alpha-amylases include the ones disclosed in WO 2005/003311 or U.S. Patent Publication no. 2005/0054071 (Novozymes) or U.S. application No. 60/638,614 (Novozymes) which is hereby incorporated by reference. A hybrid alpha-amylase may comprise an alpha-amylase catalytic domain (CD) and a carbohydrate-binding domain/module (CBM) and optional a Sinker.

Specific examples of contemplated hybrid alpha-amylases include those disclosed in U.S. application No. 60/638,614 including Fungamyl variant with catalytic domain JA118 and Athelia rolfsii SBD (SEQ ID NO: 100 in U.S. application no, 60/638,614), Rhizomucor pusillus alpha-amylase with Athelia rolfsii AMG linker and SBD (SEQ ID NO: 101 in U.S. application No. 60/638,614) and Meripilus giganteus alpha-amylase with Athelia rolfsii glucoamylase linker and SBD (SEQ ID NO: 102 in U.S. application No. 60/638,614).

Other specific examples of contemplated hybrid alpha-amylases include those disclosed in U.S. Application Publication no. 2005/0054071, including those disclosed in Table 3 on page 15, such as Aspergillus niger alpha-amylase with Aspergillus kawachii linker and starch binding domain.

### Commercial Alpha-Amylase Products

Commercial compositions comprising alpha-amylase include MYCOLASE™ from DSM (Gist Brocades), BAN™, TERMAMYL™ SC, FUNGAMYL™, LIQUOZYME™ X and SAN™ SUPER, SAN™ EXTRA L (Novozymes A/S) and CLARASE™ L-40,000, DEX-LO™, SPEZYME™ FRED, SPEZYME™ AA, SPEZYME™ HPA and SPEZYME™ DELTA AA (Genencor Int.), and the acid fungal alpha-amylase sold under the trade name SP288 (available from Novozymes A/S, Denmark).

An alpha-amylase may according to the invention be added in an amount of 0.1 to 10 AFAU/g DS, preferably 0.10 to 5 AFAU/g DS, especially 0.3 to 2 AFAU/g DS or 0.001 to 1 FAU-F/g DS, preferably 0.01 to 1 FAU-F/g DS.

When measured in KNU units the alpha-amylase activity is preferably present in an amount of 0.0005-5 KNU per g DS, preferably 0.001-1 KNU per g DS, such as around 0.050 KNU per g DS.

### Pullulanase

The pullulanase may be any pullulanase, preferably of bacterial origin. Pullulanases (E.C. 3.2.1.41, pullulan 6-glucano-hydrolase), are de-branching enzymes characterized by their ability to hydrolyze the alpha-1,6-glycosidic bonds in, for example, amylopectin and pullulan.

Specifically contemplated pullulanases include pullulanases from the genus Bacillus, preferably Bacillus amyloderamificans disclosed in U.S. Pat. No. 4,560,651 (hereby incorporated by reference), the pullulanase disclosed as SEQ ID NO: 2 in WO 01/151620 (hereby incorporated by reference), the Bacillus deramificans disclosed as SEQ ID NO: 4 in WO 01/151620 (hereby incorporated by reference), and the pullulanase from Bacillus acidopullulyticus disclosed as SEQ ID NO: 6 in WO 01/151620 (hereby incorporated by reference) and also described in FEMS Mic. Let. (1994) 115, 97-106.

Suitable commercially available pullulanase products include PROMOZYME D, PROMOZYME™ D2 (Novozymes A/S, Denmark), OPTIMAX L-300 (Genencor Int., USA), and AMANO 8 (Amano, Japan).

The pullulanase may according to the invention be added in an effective amount which include the preferred range from between 1-100 micro g per g DS, especially from 10-60 micro g per g DS, Pullulanase activity may be determined as NPUN. An Assay for determination of NPUN is described in the “Materials & Methods”-section below.

### Compositions

In the final aspect the invention relates to a composition comprising a combination of an alpha-amylase as described above and a carbohydrate-source generating enzyme.

More specifically the invention relates to a composition comprising
i) a carbohydrate-source generating enzyme; and
ii) an alpha-amylase selected from the group consisting of:
   (v) the alpha-amylase shown in SEQ ID NO: 2, or
      i) an allelic variant thereof having alpha-amylase activity, or
      ii) a fragment thereof having alpha-amylase activity,
   (x) the alpha-amylase having an amino acid sequence which has at least 60% identity with amino acids 1 to 435 of SEQ ID NO: 2;
   (y) the alpha-amylase which is encoded by a nucleotide sequence (i) which hybridizes under at least low stringency conditions with nucleotides 4 to 1308of SEQ ID NO: 1, or (ii) a complementary strand of (i); or
   (z) a variant comprising a conservative substitution, deletion, and/or insertion of one or more amino acids in positions 1 to 435 of SEQ ID NO: 2.

The alpha-amylase may in a preferred embodiment have at least 65% identity with the mature part of amino acids 1-435 of SEQ ID NO: 2, preferably at least 70% identity, preferably at least 80% identify, at least 85% identity, at least 90% identity, at least 95%, preferably at least 96%, more preferably at least 97%, more preferably at least 98% identity, or more preferably at least 99% identity with the mature part of amino acids 1-435 of SEQ ID NO: 2.

The carbohydrate-source generating enzyme may be any carbohydrate-source generating enzymes, preferably the ones mentioned in the “Carbohydrate-Source generating enzyme” section above.

Especially contemplated are glucoamylases selected from the group consisting of glucoamylases derived from the genus Aspergillus, preferably a strain of Aspergillus niger, Aspergillus oryzae, Aspergillus awamori, or the genus Athelia, preferably a strain of Athelia rolfsii, the genus Talaromyces, preferably a strain the Talaromyces emersonii, or the genus Rhizopus, such as a strain of Rhizopus nivius, or of the genus Humicola, preferably a strain of Humicola grisea var. thermoidea, or a strain of the genus Trametes, preferably a strain of Trametes cingulata.

In a preferred embodiment the amount to glucoamylase is adjusted so that the composition provides an amount during use of 0.001 to 10 AGU/g DS, preferably from 0.01 to 5 AGU/g DS, especially 0.1 to 0.5 AGU/g DS.

In a preferred embodiment the amount of alpha-amylase is adjusted so that the composition provides an amount during use of 0.01 to 10 AFAU/g DS, preferably 0.1 to 5 AFAU/g DS, especially 0.3 to 2 AFAU/g DS or in an amount of 0.0005-5 KNU per g DS, preferably 0.001-1 KNU per g DS, such as around 0.050 KNU per g DS.

In a preferred embodiment the alpha-amylase and carbohydrate-source generating enzyme, preferably glucoamylase is present in the composition in a ratio of between 0.1 and 10 AGU/AFAU, preferably 0.30 and 5 AFAU/AGU, especially between 0.5 and 3 AFAU/AGU.

The above composition is suitable for use in a fermentation product producing process of the invention.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and de-scribed herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties. The present invention is further described by the following examples which should not be construed as limiting the scope of the invention.

### Materials & Methods

### Glucoamylases:

Glucoamylase AN: Glucoamylase derived from Aspergillus niger disclosed in Boel et al. (EMBO J, 3(5): 1097-1102 (1984)) and available from Novozymes A/S, Denmark.

Glucoamylase TC: Glucoamylase derived from Trametes cingutata disclosed in WO 2006/069289 and disclosed in SEQ ID NO: 4 herein. The enzyme is also available from Novozymes A/S, Denmark on request.

Glucoamylase TE: Glucoamylase derived from Talaromyces emersonii and disclosed as SEQ ID NO: 7 in WO 99/28448.

Alpha-Amylase A: Hybrid alpha-amylase disclosed in SEQ ID NO: 2 and further disclosed in table 1 of Richardson et al. (The Journal of Biological Chemistry, Vol. 277, No 29, pp. 26501-26507 (2002)) as BD5088.

Yeast RED STAR™ available from Red Star/Lesaffre, USA

### Media and Reagents:

Chemicals used as buffers and substrates were commercial products of at least reagent grade.

PDA: 39 g/L Potato Dextrose Agar, 20 g/L agar, 50 ml/L glycerol

### Methods

Unless otherwise stated, DNA manipulations and transformations were performed using standard methods of molecular biology as described in Sambrook et al. 1989, Molecular cloning: A laboratory manual, Cold Spring Harbor Lab., Cold Spring Harbor, N.Y.; Ausubel, F. M. et al. (eds.) “Current protocols in Molecular Bioiogy”. John Wiley and Sons, 1995; Harwood, C. R., and Cutting, S. M. (eds.) “Molecular Biological Methods for Bacillus”. John Wiley and Sons, 1990.

### Glucoamylase Activity

Glucoamylase activity may be measured in AGI units or in Glucoamylase Units (AGU).

Glucoamylase activity (AGI)

Glucoamylase (equivalent to amyloglucosidase) converts starch into glucose. The amount of glucose is determined here by the glucose oxidase method for the activity determination. The method described in the section 76-11 Starch—Glucoamylase Method with Subsequent Measurement of Glucose with Glucose Oxidase in “Approved methods of the American Association of Cereal Chemists”, Vol. 1-2 AACC, from American Association of Cereal Chemists, 2000; ISBN 1-891127-12-8.

One glucoamylase unit (AGI) is the quantity of enzyme which will form 1 micro mole of glucose per minute under the standard conditions of the method.

### Standard Conditions/Reaction Conditions:

| Substrate: | Soluble starch, concentration approx. | |
|---|---|---|
| | 16 g dry matter/L. | |
| Buffer: | Acetate, approx. 0.04 M, pH = 4.3 | |
| pH: | 4.3 | |
| Incubation temperature: | 60° | C. |
| Reaction time: | 15 | minutes |

| | |
|---|---|
| Termination of the reaction: | NaOH to a concentration of approximately |
| | 0.2 g/L (pH~9) |
| Enzyme concentration: | 0.15-0.55 AAU/mL. |

The starch should be Lintner starch, which is a thin-boiling starch used in the laboratory as colorimetric indicator. Lintner starch is obtained by dilute hydrochloric acid treatment of native starch so that it retains the ability to color blue with iodine.

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 my, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | | |
|---|---|---|
| Substrate: | maltose 23.2 mM | |
| Buffer: | acetate 0.1 M | |
| pH: | 4.30 ± 0.05 | |
| Incubation temperature: | 37° C. ± 1 | |
| Reaction time: | 5 | minutes |
| Enzyme working range: | 0.5-4.0 AGU/mL | |
| Color reaction: | | |
| GlucDH: | 430 | U/L |
| Mutarotase: | 9 | U/L |
| NAD: | 0.21 | mM |
| Buffer: | phosphate 0.12 M; 0.15 M NaCl | |
| pH: | 7.60 ± 0.05 | |
| Incubation temperature: | 37° C. ± 1 | |
| Reaction time: | 5 | minutes |
| Wavelength: | 340 | nm |

A folder (EB-SM-0131.02/01) describing this analytical method in more detail is available on request from Novozymes A/S, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution, initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dexfrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Acid Alpha-Amylase Activity

When used according to the present invention the activity of any acid alpha-amylase may be measured in AFAU (Acid Fungal Alpha-amylase Units). Alternatively activity of acid alpha-amylase may be measured in AAU (Acid Alpha-amylase Units).

### Acid Alpha-Amylase Units (AAU)

The acid alpha-amylase activity can be measured in AAU (Acid Alpha-amylase Units), which is an absolute method. One Acid Amylase Unit (AAU) is the quantify of enzyme converting 1 g of starch (100% of dry matter) per hour under standardized conditions into a product having a transmission at 620 nm after reaction with an iodine solution of known strength equal to the one of a color reference.

### Standard Conditions/Reaction Conditions:

| Substrate: | Soluble starch. Concentration approx. | |
|---|---|---|
| | 20 g DS/L. | |
| Buffer: | Citrate, approx. 0.13 M, pH = 4.2 | |
| Iodine solution: | 40.176 g potassium iodide + 0.088 g iodine/L | |
| City water | 15°-20° dH (German degree hardness) | |
| pH: | 4.2 | |
| Incubation temperature: | 30° | C. |
| Reaction time: | 11 | minutes |
| Wavelength: | 620 | nm |

| | |
|---|---|
| Enzyme concentration: | 0.13-0.19 AAU/ml |
| Enzyme working range: | 0.13-0.19 AAU/mL |

The starch should be Lintner starch, which is a thin-boiling starch used in the laboratory as colorimetric indicator. Lintner starch is obtained by dilute hydrochloric acid treatment of native starch so that it retains the ability to color blue with iodine. Further detains can be found in BP 0140410 B2, which disclosure is hereby included by reference.

### Determination of FAU-F

FAU(F) Fungal Alpha-Amylase Units (Fungamyl) is measured relative to an enzyme standard of a declared strength.

Reaction conditions

| Temperature | 37° | C. |
|---|---|---|
| pH | 7.15 | |
| Wavelength | 405 | nm |
| Reaction time | 5 | min |
| Measuring time | 2 | min |

A folder (EB-SM-0216.02) describing this standard method in more detail is available on request from Novozymes A/S, Denmark, which folder is hereby included by reference.

### Acid Alpha-Amylase Activity (AFAU)

Acid alpha-amylase activity may be measured in AFAU (Acid Fungal Alpha-amylase Units), which are determined relative to an enzyme standard, 1 AFAU is defined as the amount of enzyme which degrades 5,260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glueosidic bonds in the inner regions of the starch molecule to form dexfrins and oligosaccharides with different chain lengths. The intensity of color formed with iodine is directly proportional to the concentration of starch. Amylase activity is determined using reverse colorimetry as a reduction in the concentration of starch under the specified analytical conditions,

$$STARCH + IODINE {{}^{ALPHA \text{-} AMYLASE}}_{{40}^{∘} , pH 2 , 5} DEXTRINS + OLIGOSACCHARIDES$$

$$λ = 590 nm \\ blue \text{/} violet t = 23 sec . decoloration$$

### Standard Conditions/Reaction Conditions:

| Substrate: | Soluble starch, approx. 0.17 g/L | |
|---|---|---|
| Buffer: | Citrate, approx. 0.03 M | |
| Iodine (I2): | 0.03 | g/L |
| CaCl2: | 1.85 | mM |
| pH: | 2.50 ± 0.05 | |
| Incubation temperature: | 40° | C. |
| Reaction time: | 23 | seconds |
| Wavelength: | 590 | nm |
| Enzyme concentration: | 0.025 | AFAU/mL |
| Enzyme working range: | 0.01-0.04 | AFAU/mL |

A folder EB-SM-0259.02/01 describing this analytical method in more detail is available upon request to Novozymes A/S, Denmark, which folder is hereby included by reference.

Determination of Maltogenic Amylase activity (MANU)

One MANU (Maltogenic Amylase Novo Unit) may be defined as the amount of enzyme required to release one micro mole of maltose per minute at a concentration of 10 mg of maltotriose (Sigma M 8378) substrate per ml of 0.1 M citrate buffer, pH 5.0 at 37° C. for 30 minutes.

### Determination of Pullulanase Activity (NPUN)

Endo-pullulanase activity in NPUN is measured relative to a Novozymes pullulanase standard. One pullulanase unit (NPUN) is defined as the amount of enzyme that releases 1 micro mol glucose per minute under the standard conditions (0.7% red pullulan (Megazyme), pH 5, 40° C., 20 minutes). The activity is measured in NPUN/ml using red pullulan.

1 ml diluted sample or standard is incubated at 40° C. for 2 minutes, 0.5 ml 2% red pullulan, 0.5 U KCl, 50 mM citric acid, pH 5 are added and mixed. The tubes are incubated at 40° C. for 20 minutes and stopped by adding 2.5 ml 80% ethanol. The tubes are left standing at room temperature for 10-60 minutes followed by centrifugation 10 minutes at 4000 rpm. OD of the supernatants is then measured at 510 nm and the activity calculated using a standard curve.

### EXAMPLES

### Example 1

### Yeast Propagation

Yeast is propagated prior to fermentation. Corn is ground to pass through #45 mesh screen. 200 ml tap water and 1 g urea are mixed with 300 g corn mash. Penicillin is added to 3 mg/liter. In 50 g of the mash slurry, 6.4 microL Glucoamylase AN and 0.024 g dry yeast (RED STAR™) are added and the pH is adjusted to around 5.0. The yeast slurry is incubated at 32° C. with constant stirring at 300 rpm for 7 hours in a partially open flask.

one-step fermentation Using Alpha-Amylase a Disclosed in SEQ ID NO: 2 and Glucoamylase TC

All one step ground corn to ethanol treatments are evaluated via mini-scale fermentations. Briefly, 410 g of ground corn (with particle size around 0.5 mm) is added to 590 g tap water. This mixture is supplemented with 3.0 ml 1 g/L penicillin and 1 g of urea. The pH of this slurry is adjusted to 4.5 with 5 N NaOH or diluted H₂SO₄. DS level is adjusted to around 35 wt-%. Approximately 5 g of this slurry is added to 20 ml vials. Each vial is dosed with the appropriate amount of enzyme as set out in the table below followed by addition of 200 microL yeast propagate per 5 g slurry. Actual enzyme dosages are based on the exact weight of corn slurry in each vial. Vials are closed and incubated at 32° C. immediately. 9 replicate fermentations of each treatment are run. Three replicates are selected for 24 hours, 48 hours and 70 hours time point analysis. Vials are vortexed at 24, 48 and 70 hours and analyzed by HPLC. The HPLC preparation consists of stopping the reaction by addition of 50 microL of 40% H₂SO₄, centrifuging, and filtering through a 0.45 micrometer filter. Samples are stored at 4° C. prior to analysis.

Agilent™ 1100 HPLC system coupled with RI detector is used to determine ethanol and sugars. The HPLC system consists of a degasser, quat-pump, cooled autosampler and heated column compartment. The separation column may be aminex HPX-87H ion exclusion column (300 mm×7.8 mm) from BioRad™, which links to 30 mm×4.8 mm micro-guard cation-H cartridge guard column. A 10 microL sample is injected at the flow rate of 0.6 ml/min. The mobile phase is 5 mM H₂SO₄. The column is kept at 65° C. and RI detector at 50° C. The total run time is 25 minutes per sample.

The ethanol yields after 24, 48 and 70 hours are determined.

| | Glucoamylase | Glucoamylase | Alpha-Amylase A disclosed | |
|---|---|---|---|---|
| | AN | TC | in SEQ ID NO: 2 | |
| | (AGU/g DS) | (AGU/g DS) | (AFAU/g DS) | (FAU-F/g DS) |
| 1 | — | — | 0.1 | 0.19 |
| 2 | — | — | 0.075 | 0.143 |
| 3 | — | — | 0.050 | 0.095 |
| 4 | — | — | 0.025 | 0.048 |
| 5 | — | 0.10 | 0.1 | 0.19 |
| 6 | — | 0.25 | 0.075 | 0.143 |
| 7 | — | 0.50 | 0.050 | 0.095 |
| 8 | — | 1.00 | 0.025 | 0.048 |
| 9 | 0.5 | — | 0.1 | 0.19 |
| 10 | 0.75 | — | 0.050 | 0.95 |
| 11 | 1.00 | — | 0.040 | 0.076 |
| 12 | 1.50 | — | 0.025 | 0.48 |

### Example 2

### Liquefaction and SSF Using Alpha-Amylase A Disclosed in SEQ ID NO: 2

Ground corn is used to make a 30 wt-% slurry with tap water. The pH is adjusted to approximately 5.0 using NaOH or diluted H₂SO₄. 50 NU/g DS Alpha-Amylase A is added and kept at 85° C. for 1.5 hours.

SSF is done as mini-scale fermentations. If needed, the pH is adjusted to 5.0, e.g., with diluted H₂SO₄. Mash is adjusted to a 0.5 g/L concentration Urea and 3 mg/L Penicillin. Approximately 4 grams of mash is added to 18 ml polystyrene tubes (Falcon 352025), Tubes are then dosed with the appropriate amount of Glucoamylase TE (0.3 AGU/g DS). After dosing the tubes with enzyme, they are inoculated with 0.04 ml/g mash of yeast propagate (RED STAR™) that is grown for 21 hours on corn mash. Vials are capped with a screw on lid which is punctured with a needle to allow gas release and vortexed briefly before weighing and incubation at 32° C. Fermentation progress is followed by weighing the tubes over time. Tubes are vortexed briefly before each weighing. Weight loss values are converted to ethanol yield (g ethanol/g DS) by the following formula:

$$gethanol g D S = \frac{g C {O}_{2} weightloss \times \begin{matrix}\begin{matrix}\frac{1 mol C {O}_{2}}{440098 g C {O}_{2}} \times \\ \frac{1 molethanol}{1 mol C {O}_{2}} \times \end{matrix} \\ \frac{46094 g ethanol}{1 molethanol}\end{matrix}}{g cornintube \times \% D S of corn}$$

After 70 hours of fermentation, replicates from fermentation are sacrificed for HPLC analysis for residual sugar and glycerol concentrations. The reactions are stopped by adding 30 MicroL 40% H₂SO₄ to each. The tubes are centrifuged at 3000 rpm for 15 minutes to clear the supernatant, and then 1 ml of cleared supernatant is passed through a 0.45 micron syringe filter and placed in HPLC vials. The samples are analyzed by using an Agilent HPLC System using analytical BIO-RAD Aminex HPX-87H column and a BIO-RAD Cation H refill guard column. HPLC run conditions are: 0.005M H₂SO₄ mobile phase, flow rate of 0.6 ml/min, column temperature at 65° C., RI detector (Refractive Index) at 50° C., injection volume of 10 ml, and a 25 min run time.

1-24. (canceled)

25. A process for producing a fermentation product from starch-containing material comprising the steps of:
(a) liquefying starch-containing material with an alpha-amylase;
(b) saccharifying the liquefied material using a carbohydrate-source generating enzyme;
(c) fermenting using a fermenting organism;
wherein the alpha-amylase used in the liquefaction step (a) is selected from the group consisting of:
(v) the alpha-amylase shown in SEQ ID NO: 2, or
i) an allelic variant thereof having alpha-amylase activity, or
ii) a fragment thereof having alpha-amylase activity;
(x) an alpha-amylase having an amino acid sequence which has at least 60% identity with amino acids 1 to 435 of SEQ ID NO: 2;
(y) an alpha-amylase which is encoded by a nucleotide sequence (i) which hybridizes under at least low stringency conditions with nucleotides 4 to 1308 of SEQ ID NO: 1, or (ii) a complementary strand of (i); or
(z) a variant comprising a conservative substitution, deletion, and/or insertion of one or more amino acids in positions 1 to 435 of SEQ ID NO: 2.

26. The process of claim 1, wherein one or more carbohydrases selected from the group consisting of alpha-amylase, pullulanase, and beta-amylase, or a combination thereof, is introduced during step (a).

27. The process of claim 25, wherein the alpha-amylase comprises an amino acid sequence which has at least 99% identity with amino acids 1-435 of SEQ ID NO: 2.

28. The process of claim 25, wherein the alpha-amylase is derived from a microorganism of the order Thermococcales.

29. The process of claim 25, wherein the carbohydrate-source generating enzyme is a glucoamylase, beta-amylase or maltogenic amylase, or a mixture thereof.

30. The process of claim 25, wherein the fermentation product is ethanol.

31. The process of claim 25, wherein the step (b) and (c) are carried out sequentially or simultaneously.

32. A process for producing a fermentation product from starch-containing material comprising:
(a) saccharifying starch-containing material with an alpha-amylase at a temperature below the initial gelatinization temperature of said starch-containing material;
(b) fermenting using a fermenting organism;
wherein the alpha-amylase used in liquefaction saccharification step (a) or simultaneous saccharification and fermentation in combined step (a) and (b) is selected from the group consisting of:
(v) the alpha-amylase shown in SEQ ID NO: 2, or
i) an allelic variant thereof having alpha-amylase activity, or
ii) a fragment thereof having alpha-amylase activity;
(x) an alpha-amylase having an amino acid sequence which has at least 60% identity with amino acids 1 to 435 of SEQ ID NO: 2;
(y) an alpha-amylase which is encoded by a nucleotide sequence (i) which hybridizes under at least low stringency conditions with nucleotides 4 to 1308 of SEQ ID NO: 1, or (ii) a complementary strand of (i); or
(z) a variant comprising a conservative substitution, deletion, and/or insertion of one or more amino acids in positions 1 to 435 of SEQ ID NO: 2.

33. A process of claim 32, further wherein an acid fungal alpha-amylase or a plant alpha-amylase is introduced during fermentation or simultaneous saccharification and fermentation.

34. The process of claim 32, wherein the alpha-amylase comprises an amino acid sequence which has at least 99% identity with the amino acids 1-435 of SEQ ID NO: 2.

35. The process of claim 32, wherein the alpha-amylase is derived from a microorganism of the order Thermococcales.

36. The process of claim 32, wherein the saccharification and fermentation is carried out sequentially or simultaneously.

37. The process of claim 32, wherein the temperature during saccharification in step (a) is in the range from 30° C. to 75° C.

38. The process of claim 32, wherein the temperature during simultaneous saccharification and fermentation, or fermentation in step (b), is between 28° C. and 36° C.

39. The process of claim 32, wherein a carbohydrate-source generating enzyme is present during saccharification in step (a) or simultaneous saccharification and fermentation in combined steps (a) and (b).

40. The process of claim 32, wherein the starch-containing material is uncooked granular starch.

41. The process of claim 32, wherein the fermentation product is ethanol.
